(19) United States
(12) Patent Application Publication
(10) Pub. No.: US 2008/0255128 A1
(43) Pub. Date: Oct. 16, 2008

(54) PHTHALAZINONE DERIVATIVES

(76) Inventors: Muhammad Hashim Javaid, Cambridge (GB); Marc Geoffrey Hummersone, Cambridge (GB); Keith Allan Menear, Cambridge (GB); Sylvie Gomez, Cambridge (GB); Niall Morrison Barr Martin, Cambridge (GB)

Correspondence Address: MICHAEL BEST & FRIEDRICH LLP, ONE SOUTH PINCKNEY STREET, P O BOX 1806, MADISON, WI, 53701, US

(21) Appl. No.: 12/100,614

(22) Filed: Apr. 10, 2008

Related U.S. Application Data
(60) Provisional application No. 60/910,887, filed on Apr. 10, 2007.

(51) Int. Cl. A61K 31/502; C07D 237/30; A61P 35/04; A61P 3/00; A61P 9/00; A61P 19/00; A61P 25/00

(52) U.S. Cl. 514/248; 544/237

(57) ABSTRACT

A compound of the formula (I):

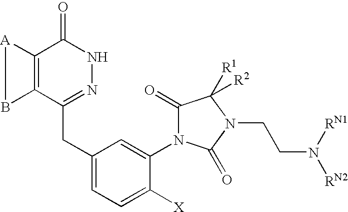

wherein:
A and B together represent an optionally substituted, fused aromatic ring;
X is selected from H and F;
R¹ and R² are independently selected from H and methyl;
R^{N1} is selected from H and optionally substituted C₁₋₇ alkyl;
R^{N2} is selected from H, optionally substituted C₁₋₇ alkyl, C₃₋₇ heterocyclyl and C₅₋₆ aryl;
or R^{N1} and R^{N2} and the nitrogen atom to which they are bound form an optionally substituted nitrogen containing C₅₋₇ heterocyclic group.

This application claims priority to U.S. Provisional Application No. 60/910,887, filed Apr. 10, 2007, which is incorporated herein by reference in its entirety.

The present invention relates to phthalazinone derivatives and their use as pharmaceuticals. In particular, the present invention relates to the use of these compounds to inhibit the activity of the enzyme poly (ADP-ribose)polymerase-1, also known as poly(ADP-ribose)synthase and poly ADP-ribosyltransferase, and commonly referred to as PARP-1.

The mammalian enzyme PARP-1 (a 113-kDa multidomain protein) has been implicated in the signalling of DNA damage through its ability to recognize and rapidly bind to DNA single or double strand breaks (D'Amours, et al., Biochem. J., 342, 249-268 (1999)).

The family of Poly (ADP-ribose) polymerases now includes around 18 proteins, that all display a certain level of homology in their catalytic domain but differ in their cellular functions (Ame et al., Bioessays., 26(8), 882-893 (2004)). Of this family PARP-1 (the founding member) and PARP-2 are so far the sole enzymes whose catalytic activity are stimulated by the occurrence of DNA strand breaks, making them unique in the family.

It is now known that PARP-1 participates in a variety of DNA-related functions including gene amplification, cell division, differentiation, apoptosis, DNA base excision repair as well as effects on telomere length and chromosome stability (d'Adda di Fagagna, et al., Nature Gen., 23(1), 76-80 (1999)).

Studies on the mechanism by which PARP-1 modulates DNA repair and other processes has identified its importance in the formation of poly (ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP-1 utilizes NAD⁺ to synthesize poly (ADP-ribose) on a variety of nuclear target proteins, including topoisomerases, histones and PARP itself (Rhun, et al., Biochem. Biophys. Res. Commun., 245, 1-10 (1998))

Poly (ADP-ribosyl)ation has also been associated with malignant transformation. For example, PARP-1 activity is higher in the isolated nuclei of SV40-transformed fibroblasts, while both leukaemic and colon cancer cells show higher enzyme activity than the equivalent normal leukocytes and colon mucosa (Miwa, et al., Arch. Biochem. Biophys., 181, 313-321 (1977); Burzio, et al., Proc. Soc. Exp. Biol. Med., 149, 933-938 (1975); and Hirai, et al., Cancer Res., 43, 3441-3446 (1983)). More recently in malignant prostate tumours compared to benign prostate cells significantly increased levels of active PARP (predominantly PARP-1) have been identified associated with higher levels of genetic instability (McNealy, et al., Anticancer Res., 23, 1473-1478 (2003)).

A number of low-molecular-weight inhibitors of PARP-1 have been used to elucidate the functional role of poly (ADP-ribosyl)ation in DNA repair. In cells treated with alkylating agents, the inhibition of PARP leads to a marked increase in DNA-strand breakage and cell killing (Durkacz, et al., Nature, 283, 593-596 (1980); Berger, N. A., Radiation Research, 101, 4-14 (1985)).

Subsequently, such inhibitors have been shown to enhance the effects of radiation response by suppressing the repair of potentially lethal damage (Ben-Hur, et al., British Journal of Cancer, 49 (Suppl. VI), 34-42 (1984); Schlicker, et al., Int. J. Radiat. Biol., 75, 91-100 (1999)). PARP inhibitors have been reported to be effective in radio sensitising hypoxic tumour cells (U.S. Pat. No. 5,032,617; U.S. Pat. No. 5,215,738 and U.S. Pat. No. 5,041,653). In certain tumour cell lines, chemical inhibition of PARP-1 (and PARP-2) activity is also associated with marked sensitisation to very low doses of radiation (Chalmers, Clin. Oncol., 16(1), 29-39 (2004))

Furthermore, PARP-1 knockout (PARP −/−) animals exhibit genomic instability in response to alkylating agents and γ-irradiation (Wang, et al., Genes Dev., 9, 509-520 (1995); Ménissier-de Murcia, et al., Proc. Natl. Acad. Sci. USA, 94, 7303-7307 (1997)). More recent data indicates that PARP-1 and PARP-2 possess both overlapping and non-redundant functions in the maintenance of genomic stability, making them both interesting targets (Ménissier-de Murcia, et al., EMBO. J., 22(9), 2255-2263 (2003)).

PARP inhibition has also recently been reported to have antiangiogenic effects. Where dose dependent reductions of VEGF and basic-fibroblast growth factor (bFGF)-induced proliferation, migration and tube formation in HUVECS has been reported (Rajesh, et al., Biochem. Biophys. Res. Comm., 350, 1056-1062 (2006)).

A role for PARP-1 has also been demonstrated in certain vascular diseases, septic shock, ischaemic injury and neurotoxicity (Cantoni, et al., Biochim. Biophys. Acta, 1014, 1-7 (1989); Szabo, et al., J. Clin. Invest., 100, 723-735 (1997)). Oxygen radical DNA damage that leads to strand breaks in DNA, which are subsequently recognised by PARP-1, is a major contributing factor to such disease states as shown by PARP-1 inhibitor studies (Cosi, et al., J. Neurosci. Res., 39, 38-46 (1994); Said, et al., Proc. Natl. Acad. Sci. U.S.A., 93, 4688-4692 (1996)). More recently, PARP has been demonstrated to play a role in the pathogenesis of haemorrhagic shock (Liaudet, et al., Proc. Natl. Acad. Sci. U.S.A., 97(3), 10203-10208 (2000)), eye (Occular) related oxidative damage as in Macular Degeneration (AMD) and retinitis pigmentosis (Paquet-Durand et al., J. Neuroscience, 27(38), 10311-10319 (2007), as well as in transplant rejection of organs like lung, heart and kidney (O'Valle, et al., Transplant. Proc., 39(7), 2099-2101 (2007). Moreover, treatment with PARP inhibitors has been shown to attenuate acute diseases like pancreatitis and it associated liver and lung damage caused by mechanisms where PARP plays a role (Mota, et al., Br. J. Pharmacol., 151(7), 998-1005 (2007).

It has also been demonstrated that efficient retroviral infection of mammalian cells is blocked by the inhibition of PARP-1 activity. Such inhibition of recombinant retroviral vector infections was shown to occur in various different cell types (Gaken, et al., J. Virology, 70(6), 3992-4000 (1996)). Inhibitors of PARP-1 have thus been developed for the use in anti-viral therapies and in cancer treatment (WO 91/18591).

Moreover, PARP-1 inhibition has been speculated to delay the onset of aging characteristics in human fibroblasts (Rattan and Clark, Biochem. Biophys. Res. Comm., 201(2), 665-672 (1994)) and age related diseases such as atherosclerosis (Hans, et al., Cardiovasc. Res., (Jan. 31, 2008)). This may be related to the role that PARP plays in controlling telomere function (d'Adda di Fagagna, et al., Nature Gen., 23(1), 76-80 (1999)).

PARP inhibitors are also thought to be relevant to the treatment of inflammatory bowel disease (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204), ulcerative colitis (Zingarelli, B, et al., Immunology, 113(4), 509-517 (2004)) and Crohn's disease (Jijon, H. B., et al., Am. J. Physiol. Gastrointest. Liver Physiol., 279, G641-G651 (2000).

Some of the present inventors have previously described (WO 02/36576) a class of 1(2H)-phthalazinone compounds which act as PARP inhibitors. The compounds have the general formula:

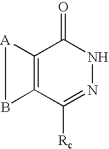

where A and B together represent an optionally substituted, fused aromatic ring and where Rc is represented by -L-R_{L}. A large number of examples are of the formula:

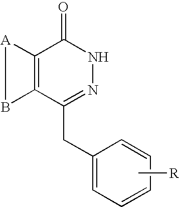

where R represent one or more optional substituents.

Some of the present inventors described a particular class of the above compounds in WO 03/093261, which have the general formula as above, and wherein R is in the meta position, and the examples disclosed have the R group selected from:

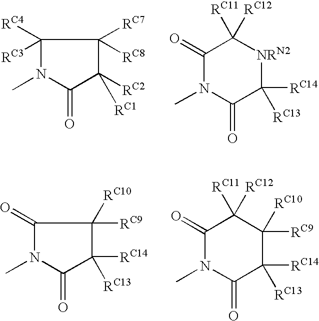

The present inventors have now discovered that compounds with a different substituent groups to those above exhibit surprising levels of inhibition of the activity of PARP, and/or of potentiation of tumour cells to radiotherapy and various chemotherapies. In addition, the stability of the compounds of the present invention is in general improved over those compounds exemplified in WO 03/093261. Some of the compounds of the present invention also show good solubility in both aqueous media and phosphate buffer solution—enhanced solubility may be of use in formulation the compounds for administration by an IV route, or for oral formulations (e.g. liquid and small tablet forms) for paediatric use. The oral bioavailablity of the compounds of the present invention may be enhanced.

Further compounds related to those exemplified in WO 03/093261 are disclosed in co-pending U.S. application Ser. No. 11/550,004 and co-pending PCT application published as WO 2007/045877.

Accordingly, the first aspect of the present invention provides a compound of the formula (I):

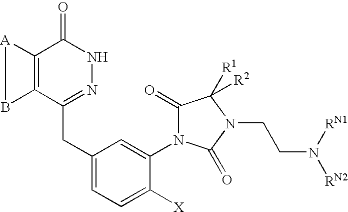

(including isomers, salts, solvates, chemically protected forms, and prodrugs thereof)

wherein:

A and B together represent an optionally substituted, fused aromatic ring;

X is selected from H and F;

R¹ and R² are independently selected from H and methyl;

R^{N1} is selected from H and optionally substituted C₁₋₇ alkyl;

R^{N2} is selected from H, optionally substituted C₁₋₇ alkyl, C₃₋₇ heterocyclyl and C₅₋₆ aryl;

or R^{N1} and R^{N2} and the nitrogen atom to which they are bound form an optionally substituted nitrogen containing C₅₋₇ heterocyclic group.

A second aspect of the present invention provides a pharmaceutical composition comprising a compound of the first aspect and a pharmaceutically acceptable carrier or diluent.

A third aspect of the present invention provides the use of a compound of the first aspect in a method of treatment of the human or animal body.

A fourth aspect of the present invention provides the use of a compound as defined in the first aspect of the invention in the preparation of a medicament for:

(a) preventing poly(ADP-ribose) chain formation by inhibiting the activity of cellular PARP (PARP-1 and/or PARP-2);

(b) the treatment of: vascular disease; septic shock; ischaemic injury, both cerebral and cardiovascular; reperfusion injury, both cerebral and cardiovascular; neurotoxicity, including acute and chronic treatments for stroke and Parkinsons disease; angiogenesis; haemorraghic shock; eye related oxidative damage; transplant rejection; inflammatory diseases, such as arthritis, inflammatory bowel disease, ulcerative colitis and Crohn's disease; multiple sclerosis; secondary effects of diabetes; as well as the acute treatment of cytoxicity following cardiovascular surgery; pacreatitis; atherosclerosis; or diseases ameliorated by the inhibition of the activity of PARP;

(c) use as an adjunct in cancer therapy or for potentiating tumour cells for treatment with ionizing radiation or chemotherapeutic agents.

In particular, compounds as defined in the first aspect of the invention can be used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. Such combinations could be given, for example, as intravenous preparations or by oral administration as dependent on the preferred method of administration for the particular agent.

Other further aspects of the invention provide for the treatment of disease ameliorated by the inhibition of PARP, comprising administering to a subject in need of treatment a therapeutically-effective amount of a compound as defined in the first aspect, preferably in the form of a pharmaceutical composition and the treatment of cancer, comprising administering to a subject in need of treatment a therapeutically-effective amount of a compound as defined in the first aspect in combination, preferably in the form of a pharmaceutical composition, simultaneously or sequentially with radiotherapy (ionizing radiation) or chemotherapeutic agents.

In further aspects of the present invention, the compounds may be used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, comprising administering to said patient a therapeutically-effective amount of the compound.

The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_{—}000051), RAD51 (NM_{—}002875), RAD51L1 (NM_{—}002877), RAD51C (NM_{—}002876), RAD51L3 (NM_{—}002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE11A (NM_{—}005590) and NBS1 (NM_{—}002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001).

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some preferred embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535) or by an epigenetic mechanism such as gene promoter methylation.

BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol Med., 8(12), 571-6, (2002)). The association of BRCA1 and/or BRCA2 mutations with breast cancer is well-characterised in the art (Radice, P. J., Exp Clin Cancer Res., 21(3 Suppl), 9-12 (2002)). Amplification of the EMSY gene, which encodes a BRCA2 binding factor, is also known to be associated with breast and ovarian cancer.

Carriers of mutations in BRCA1 and/or BRCA2 are also at elevated risk of cancer of the ovary, prostate and pancreas.

In some preferred embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

### FIGURES

FIG. 1 is an X-ray diffraction pattern of a crystalline form of a compound of the present invention.

FIG. 2 is a DSC thermogram of the same crystalline form.

FIG. 3 is a TGA thermogram of the same crystalline form.

### DEFINITIONS

The term “aromatic ring” is used herein in the conventional sense to refer to a cyclic aromatic structure, that is, a cyclic structure having delocalised π-electron orbitals.

The aromatic ring fused to the main core, i.e. that formed by -A-B—, may bear further fused aromatic rings (resulting in, e.g. naphthyl or anthracenyl groups). The aromatic ring(s) may comprise solely carbon atoms, or may comprise carbon atoms and one or more heteroatoms, including but not limited to, nitrogen, oxygen, and sulfur atoms. The aromatic ring(s) preferably have five or six ring atoms.

The aromatic ring(s) may optionally be substituted. If a substituent itself comprises an aryl group, this aryl group is not considered to be a part of the aryl group to which it is attached. For example, the group biphenyl is considered herein to be a phenyl group (an aryl group comprising a single aromatic ring) substituted with a phenyl group. Similarly, the group benzylphenyl is considered to be a phenyl group (an aryl group comprising a single aromatic ring) substituted with a benzyl group.

In one group of preferred embodiments, the aromatic group comprises a single aromatic ring, which has five or six ring atoms, which ring atoms are selected from carbon, nitrogen, oxygen, and sulfur, and which ring is optionally substituted. Examples of these groups include, but are not limited to, benzene, pyrazine, pyrrole, thiazole, isoxazole, and oxazole. 2-Pyrone can also be considered to be an aromatic ring, but is less preferred.

If the aromatic ring has six atoms, then preferably at least four, or even five or all, of the ring atoms are carbon. The other ring atoms are selected from nitrogen, oxygen and sulphur, with nitrogen and oxygen being preferred. Suitable groups include a ring with: no hetero atoms (benzene); one nitrogen ring atom (pyridine); two nitrogen ring atoms (pyrazine, pyrimidine and pyridazine); one oxygen ring atom (pyrone); and one oxygen and one nitrogen ring atom (oxazine).

If the aromatic ring has five ring atoms, then preferably at least three of the ring atoms are carbon. The remaining ring atoms are selected from nitrogen, oxygen and sulphur. Suitable rings include a ring with: one nitrogen ring atom (pyrrole); two nitrogen ring atoms (imidazole, pyrazole); one oxygen ring atom (furan); one sulphur ring atom (thiophene); one nitrogen and one sulphur ring atom (isothiazole, thiazole); and one nitrogen and one oxygen ring atom (isoxazole or oxazole).

The aromatic ring may bear one or more substituent groups at any available ring position. These substituents are selected from halo, nitro, hydroxy, ether, thiol, thioether, amino, C₁₋₇ alkyl, C₃₋₂₀ heterocyclyl and C₅₋₂₀ aryl. The aromatic ring may also bear one or more substituent groups which together form a ring. In particular these may be of formula —(CH₂)ₘ— or —O—(CH₂)ₚ—O—, where m is 2, 3, 4 or 5 and p is 1, 2 or 3.

Nitrogen-containing C₅₋₇ heterocyclylic ring: The term “nitrogen-containing C₅₋₇ heterocyclylic ring” as used herein, pertains to a C₅₋₇ heterocyclylic ring, as defined below with relation to heterocyclyl, having at least one nitrogen ring atom.

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include, but are not limited to, iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C≡CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of cycloalkyl groups include, but are not limited to, those derived from:
saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);
unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);
saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);
unsaturated polycyclic hydrocarbon compounds:

camphene (C₁₀), limonene (C₁₀), pinene (C₁₀);
polycyclic hydrocarbon compounds having an aromatic ring:

indene (Cg), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The term “C₅₋₆ aryl” as used herein, pertains to a monovalent moiety obtained by removing a hydrogen atom from an aromatic ring atom of a C₅₋₆ aromatic compound, said compound having one aromatic ring having 5 or 6 ring atoms. Examples and further limitations are given above in the definition of “C₅₋₂₀ aryl”.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. In particular, the cyclic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

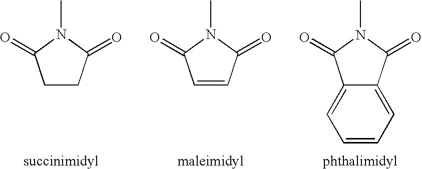

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHCONHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol: —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl(tosyl).

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

### Further Embodiments

The following embodiments can relate to each aspect of the present invention, where applicable.

In the present invention, the fused aromatic ring(s) represented by -A-B— may consist of solely carbon ring atoms, and thus may be benzene, naphthalene, and is more preferably benzene. As described above, these rings may be substituted, but in some embodiments are preferably unsubstituted.

If the fused aromatic ring represented by -A-B— bears one or more substituent groups, these are preferably attached to the atoms which themselves is attached to the central ring α- to the carbon atom in the central ring. Thus, if the fused aromatic ring is a benzene ring, the preferred places of substitution is shown in the formula below by *:

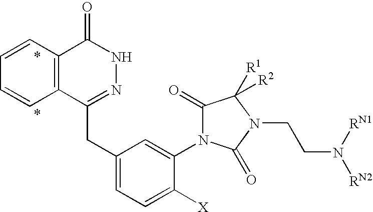

This substituent may be selected from a halo group, and more particularly F.

X is preferably F.

R¹ and R² may both be H or methyl, or R¹ and R² may be H and methyl respectively. It is preferred that R¹ and R² are H and methyl respectively.

If R^{N1} is C₁₋₇ alkyl it may be unsubstituted, for example, methyl, ethyl, cyclopropyl, iso-propyl, tert-butyl, 2,2-dimethylpropyl, cyclobutyl, cyclohexyl, or may be substituted, for example, by a group selected from halo (F), hydroxy, alkoxy (methoxy) and C₅₋₆ aryl (pyridyl, phenyl).

If R^{N2} is C₁₋₇ alkyl it may be unsubstituted, for example, methyl, ethyl, cyclopropyl, iso-propyl, tert-butyl, 2,2-dimethylpropyl, cyclobutyl, cyclohexyl, or may be substituted, for example, by a group selected from halo (F), hydroxy, alkoxy (methoxy) and C₅₋₆ aryl (pyridyl, phenyl).

If R^{N2} is C₃₋₇ heterocyclyl, then it may be substituted or unsubstituted. Substitutent may include C₁₋₇ alkyl, halo, hydroxy, alkoxy and amino. It may be a C₃, C₄, C₅, C₆ or C₇ heterocylcyl and may contain 1, 2 or 3 ring heteroatoms, and may contain unsaturation. In some embodiments, R^{N2} is a C₅₋₆ heterocyclyl, for example, 4,5-dihydro-thiazol-2-yl.

If R^{N2} is C₅₋₆ aryl, then is may be substituted or unsubstituted. Substitutent may include, C₁₋₇ alkyl, halo, hydroxy, alkoxy and amino. It may be a C₅ (pyrolyl, oxazolyl) or C₆ aryl (phenyl, pyridiyl, pyrazinyl).

R^{N1} and R^{N2} may be the same, i.e. may be selected from H and optionally substituted C₁₋₇ alkyl. In particular, when R^{N1} and R^{N2} are the same, they may be selected from unsubstituted C₁₋₇ alkyl, for example, methyl, ethyl, iso-propyl.

When R^{N2} is C₃₋₇ heterocyclyl or C₅₋₆ aryl or is C₁₇ alkyl substitued by C₅₋₆ aryl , R^{N1} may be hydrogen.

If R^{N1} and R^{N2} and the nitrogen atom to which they are bound form an optionally substituted nitrogen containing C₅₋₇ heterocyclic group, this group may be selected from pyrrolidine, piperidine, morpholine and thiomorpholine. The C₅₋₇ heterocyclic group may be substituted or unsubstituted. If the C₅₋₇ heterocyclic group is substituted, the substituents may be selected from C₁₋₇ alkyl (methyl, ethyl), C₅₋₆ aryl (furanyl), hydroxy and C₁₋₇ alkyoxy (methoxy). These substituents may be at any ring position. Examples of groups in the present invention include, but are not limited to, pyrrolidine, 2,6-dimethyl-morpholine, 1,2,3,6-tetrahydro-pyridine, 2-methyl-pyrrolidine, piperidine, morpholino, 2-methyl-piperidine, 3-hydroxy-piperidine, thiomorpholine, 2-ethyl-piperidine, 4,4-dimethyl-piperidine, 3,3-dimethyl-piperidine, 2-furan-2-yl-pyrrolidine and 2,2,6,6-tetramethyl-piperidine.

A compound of particular interest is 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (9).

Includes Other Forms

Included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

### Isomers, Salts, Solvates, Protected Forms, and Prodrugs

Certain compounds may exist in one or more particular geometric, optical, enantiomeric, diasterioisomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and /-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Particularly relevant to the present invention is the tautomeric pair illustrated below:

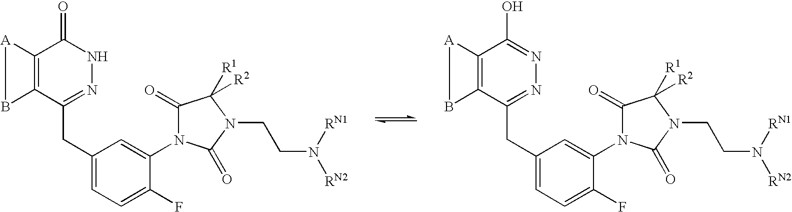

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

When R¹ is H and R² is methyl, the compounds of the present invention have a chiral centre, indicated by * in the formula below:

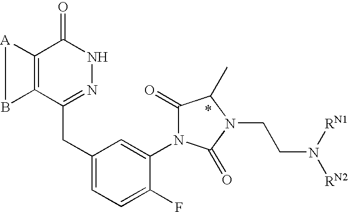

Reference to this compound includes the stereoisomeric forms, as well as (wholly or partially) racemic and other mixtures thereof.

Compound 9 once separated by chiral HPLC has been found to epimerise on standing in solution.

Unless otherwise specified, a reference to a particular compound also includes ionic, salt, solvate, and protected forms of thereof, for example, as discussed below, as well as its different polymorphic forms.

It may be convenient or desirable to prepare, purify, and/or handle a corresponding salt of the active compound, for example, a pharmaceutically-acceptable salt. Examples of pharmaceutically acceptable salts are discussed in Berge, et al., “Pharmaceutically Acceptable Salts”, J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali met al ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

Salts of particular interest in the present invention are: hydrochloride, succinate, fumarate, mesylate, tosylate, maleate, sulphate and phosphate, and in particular hydrochloride.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH—Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: a C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include, but are not limited to, those wherein R is C₁₋₂₀ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl;

(4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleaved by phosphatase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Hydrochloride Salt of Compound 9

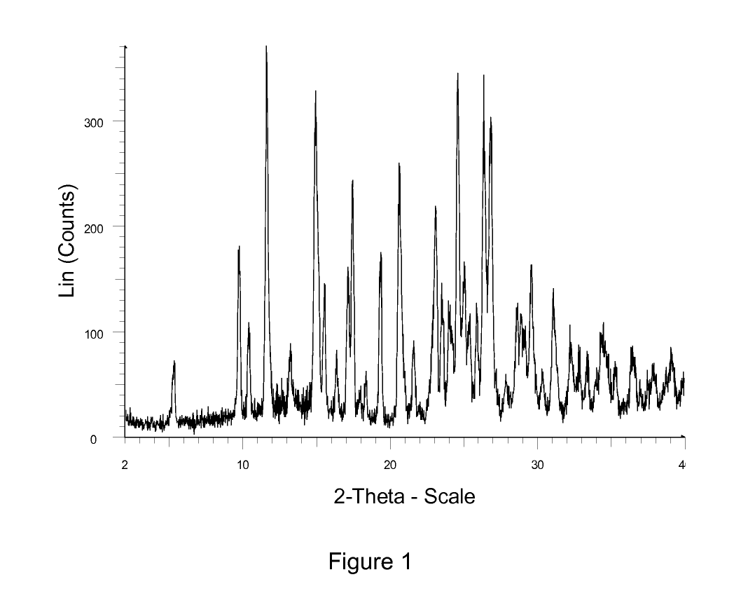

The hydrochloride salt of compound 9, and its particular crystalline form (hereinafter termed “9a form 1”), as described in the examples below, are aspects of the present invention. 9a form 1 is characterised in providing at least one of the following 20 values measured using CuKa radiation: 11.6 and 24.6°. 9a form 1 may be characterized by having an X-ray diffraction pattern comprising 2 or more of the ten most prominent peaks as set out in table B in Example 6. 9a form 1 may also be characterised in providing an X-ray powder diffraction pattern, substantially as shown in FIG. 1. The peaks may be at the stated values or within 0.5° 2 theta either side of the stated values.

When it is stated that an aspect of the present invention relates to 9a form 1, the degree of crystallinity is conveniently greater than about 60%, more conveniently greater than about 80%, preferably greater than about 90% and more preferably greater than about 95%. Most preferably the degree of crystallinity is greater than about 98%.

9a form 1 provides X-ray powder diffraction patterns substantially the same as the X-ray powder diffraction patterns shown in FIG. 1 and has substantially the ten most prominent peaks (angle 2-theta values) shown in Table B in Example 6. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the 9a form 1 of the present invention is not limited to the crystals that provide X-ray powder diffraction patterns identical to the X-ray powder diffraction pattern shown in FIG. 1, and any crystals providing X-ray powder diffraction patterns substantially the same as those shown in FIG. 1 fall within the scope of the present invention. A person skilled in the art of X-ray powder diffraction is able to judge the substantial identity of X-ray powder diffraction patterns.

Persons skilled in the art of X-ray powder diffraction will realise that the relative intensity of peaks can be affected by, for example, grains above 30 microns in size and non-unitary aspect ratios, which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values. (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

Generally, a measurement error of a diffraction angle in an X-ray powder diffractogram is approximately plus or minus 0.5° 2-theta, and such degree of a measurement error should be taken into account when considering the X-ray powder diffraction pattern in FIG. 1 and when reading Table B. Furthermore, it should be understood that intensities might fluctuate depending on experimental conditions and sample preparation (preferred orientation).

Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

Synthesis

Compounds of formula I of the present invention:

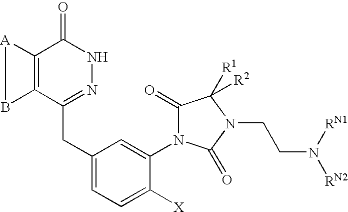

can be synthesized from compounds of formula 2:

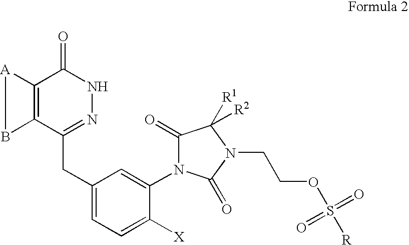

where R is a sulfone substituent, such as methyl or 4-methylphenyl, by reaction with the appropriate amine HNR^{N1}R^{N2} in an appropriate organic solvent, for example acetonitrile.

Compounds of formula 2 can be derived from compounds of formula 3:

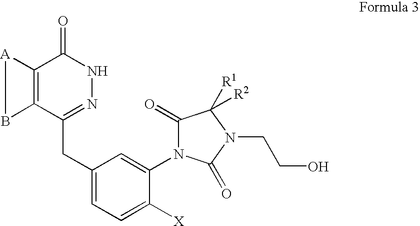

by reaction first with a base, such as triethylamine, followed by reaction with the appropriate sulfonyl chloride RSO₃Cl.

Compounds of formula 3 can be synthesized from compounds of formula 4:

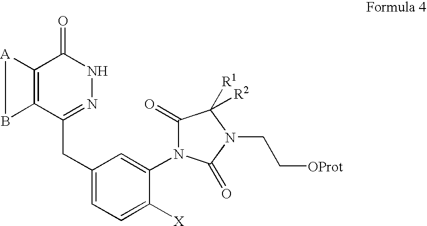

where Prot is an hydroxyl protecting group, for example, a silyl ether (TBDMS), using the appropriate deprotection conditions.

Compounds of formula 4 can be synthesized via an intermediate of formula 5:

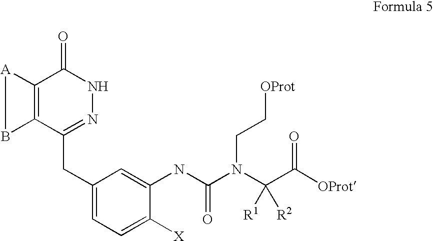

where OProt′ represents an orthogonally protected hydroxy group, for example, a C₁₋₄ alkoxy group (OEt), which is produced by the coupling a compound of formula 6:

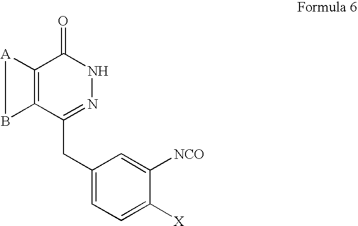

with a compound of formula 7:

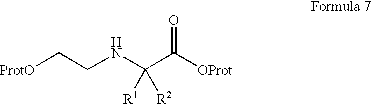

The urea bond formation reaction is carried out under standard conditions. Compounds of formulae 7 may be synthesized by the coupling of compounds of formulae 8 and 9:

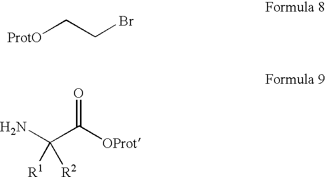

for example in the presence of potassium carbonate and Hunig's base.

The compounds of formulae 6 may be synthesized by known methods, as exemplified below.

Compounds of formula I of the present invention:

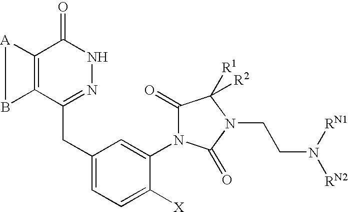

may also be synthesized from compounds of formula 6:

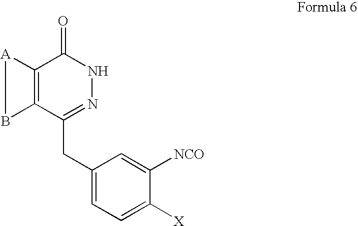

by reaction with a compound of formula 10:

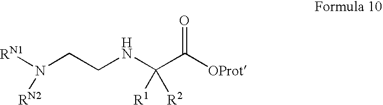

which undergoes ring closure. Compounds of formula 6 may be obtained by the Curtius reaction from the corresponding carboxylic acid.

Use

The present invention provides active compounds, specifically, active in inhibiting the activity of PARP-1.

The term “active” as used herein, pertains to compounds which are capable of inhibiting PARP-1 activity, and specifically includes both compounds with intrinsic activity (drugs) as well as prodrugs of such compounds, which prodrugs may themselves exhibit little or no intrinsic activity.

One assay which may conveniently be used in order to assess the PARP-1 inhibition offered by a particular compound is described in the examples below.

The present invention further provides a method of inhibiting the activity of PARP-1 in a cell, comprising contacting said cell with an effective amount of an active compound, preferably in the form of a pharmaceutically acceptable composition. Such a method may be practised in vitro or in vivo.

For example, a sample of cells may be grown in vitro and an active compound brought into contact with said cells, and the effect of the compound on those cells observed. As examples of “effect”, the amount of DNA repair effected in a certain time may be determined. Where the active compound is found to exert an influence on the cells, this may be used as a prognostic or diagnostic marker of the efficacy of the compound in methods of treating a patient carrying cells of the same cellular type.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “adjunct” as used herein relates to the use of active compounds in conjunction with known therapeutic means. Such means include cytotoxic regimens of drugs and/or ionising radiation as used in the treatment of different cancer types. In particular, the active compounds are known to potentiate the actions of a number of cancer chemotherapy treatments, which include the topoisomerase class of poisons and most of the known alkylating agents used in treating cancer.

Active compounds may also be used as cell culture additives to inhibit PARP, for example, in order to sensitize cells to known chemotherapeutic agents or ionising radiation treatments in vitro.

Active compounds may also be used as part of an in vitro assay, for example, in order to determine whether a candidate host is likely to benefit from treatment with the compound in question.

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutang, gibbon), or a human.

Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and 1. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

General Experimental Methods

Preparative HPLC

Instrument: Waters ZQ LC-MS system No. LAA 254 operating in Electrospray ionisation mode.

Mobile Phase A: 0.1% Formic acid in water

Mobile Phase B: 0.1% Formic acid in acetonitrile

Column: Genesis C18 4 μm 50×4.6 mm

Flow rate: 2.0 ml/min.

PDA Scan range: 210-400 nm.

Gradient 1:

| Time (mins.) | % B |
|---|---|
| 1 | 5 |
| 5 | 95 |
| 10 | 95 |
| 10.5 | 5 |
| 11 | 5 |

Gradient 2:

| Time (mins.) | % B |
|---|---|
| 1 | 5 |
| 20 | 95 |
| 23 | 95 |
| 24 | 5 |
| 25 | 5 |

NMR

¹H NMR and ¹³C NMR were typically recorded using Bruker DPX 300 spectrometer at 300 MHz and 75 MHz respectively. Chemical shifts were reported in parts per million (ppm) on the δ scale relative to tetramethylsilane internal standard. Unless stated otherwise all samples were dissolved in DMSO-d₆.

### Example 1

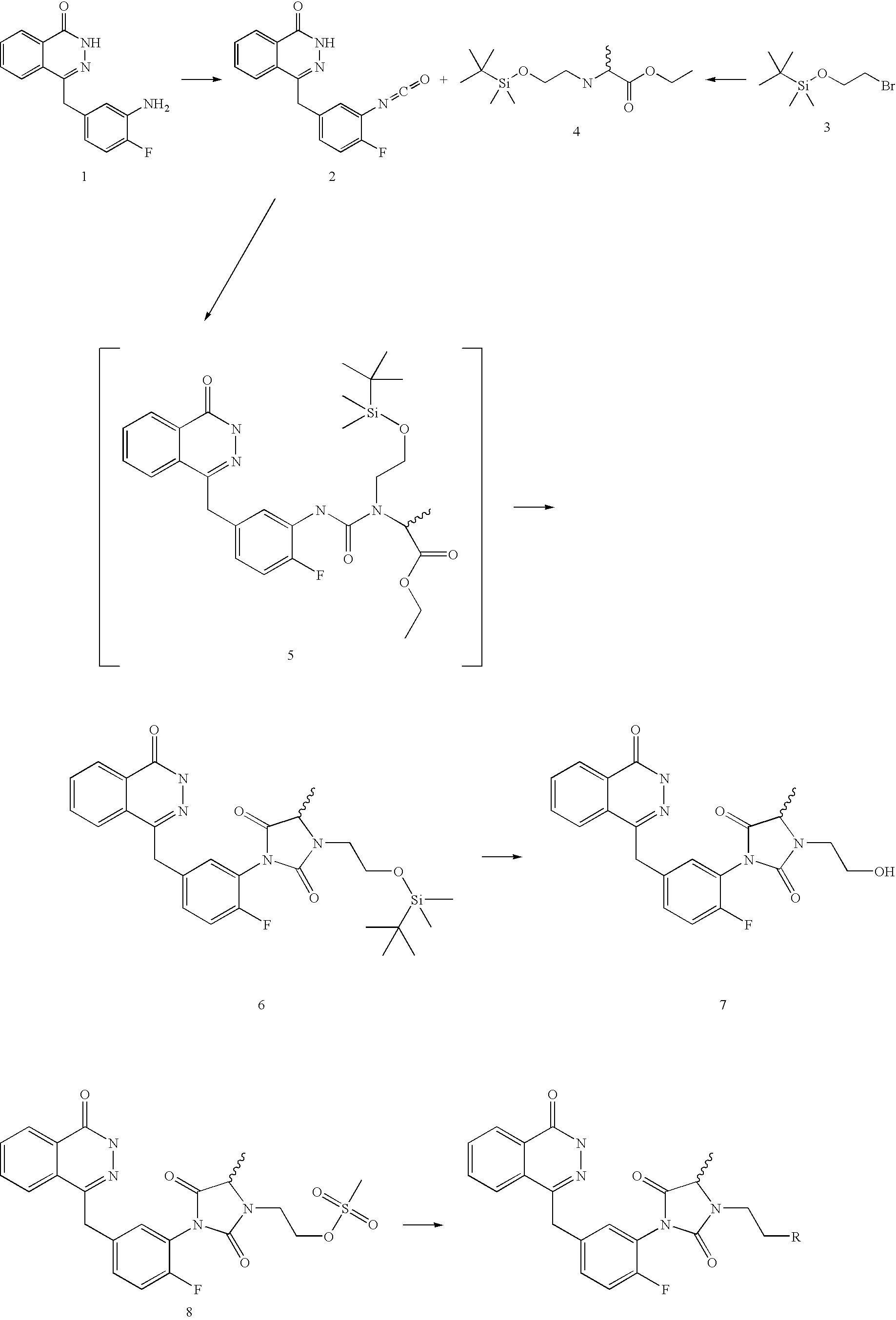

Compound 1 was sythesised as described in Example 23, of WO 03/093261, which is incorporated herein by reference.

### (a) 4-(4-Fluoro-3-isocyanato-benzyl)-2H-phthalazin-1-one (2)

To a suspension of 4-(3-amino-4-fluoro-benzyl)-2H-phthalazin-1-one (1)(4.0 g, 14.8 mmol) in anhydrous DCM (1.6 L) and triethylamine (4.62 mL, 40.86 mmol), was added a dropwise preformed solution of triphosgene (2.75 g, 9.28 mmol) in anhydrous DCM (327 mL) and stirred for 70 minutes and room temperature. The reaction mixture was then concentrated to dryness in vacuo yielding a grey solid. Single peak in LC-MS analysis, (yield taken to be quantitative) no purification performed. m/z (LC-MS, ESP), RT=4.49 mins, (M+MeOH) 328.0.

### (b) 2-[2-(tert-Butyl-dimethyl-silanyloxy)-ethylamino]-propionic acid ethyl ester (4)

To a suspension of (D/L)-alanine ethyl ester hydrochloride (19.0 mmol, 2.92 g) in DMF (100 ml) was added potassium carbonate (42.75 mmol, 5.9 g) followed by Hunig's base (7.5 ml 42.8 mmol). The mixture was then heated to 90° C. and (2-bromoethoxy)-tert butyl dimethylsilane (3)(20.9 mmol, 5.0 g) added dropwise over 2 hours. The reaction mixture was maintained at 90° C. for a further 16 hours before being cooled to room temperature. The resultant suspension was filtered and washed with DMF (2×30 ml). The filtrate was concentrated in vacuo and taken through to the next step without any need for further purification. (Rf 0.55 DCM/ethyl acetate 8:3, anisaldehyde stain).

### (c) 1-[2-(tert-Butyl-dimethyl-silanyloxy)-ethyl]-3-[2-fluoro-5-(4-oxo-3,4-dihydro phthalazin-1-ylmethyl)-phenyl]-5-methyl-imidazolidine-2,4-dione (6)

To crude 2-[2-(tert-butyl-dimethyl-silanyloxy)-ethylamino]-propionic acid ethyl ester (4)(20.9 mmol) dissolved in dry DMF (100 ml) was added magnesium sulfate (˜4.0 g). The suspension was stirred for 10 minutes and then filtered. The filtrate treated with 4-(4-fluoro-3-isocyanato-benzyl)-2H-phthalazin-1-one (2)(6.07 g, 20.9 mmol) and stirred for 18 hours at room temperature. The reaction mixture was filtered and the filtrate concentrated in vacuo to afford a crude oil. The material which was subjected to flash chromatography eluent DCM/methanol 1% initial, increasing to 2% methanol over 5 column volumes. The desired product was isolated as a brown oil. Major component in LC-MS (4.2 g, 76% purity); m/z (LC-MS, ESP), RT=4.32 mins (M+H) 525. This material was used in subsequent reactions without need for any further purification.

### (d) 3-[2-fluoro-5-(4-oxo-3,4-dihydro-phthalazin-1-ylmethyl)-phenyl]-1-(2-hydroxy-ethyl)-5-methyl-imidazolidine-2,4-dione (7)

To a solution of 1-[2-(tert-butyl-dimethyl-silanyloxy)-ethyl]-3-[2-fluoro-5-(4-oxo-3,4-dihydro phthalazin-1-ylmethyl)-phenyl]-5-methyl-imidazolidine-2,4-dione (6) (4.2 g, 76% pure) dissolved in THF (50 ml) was added TBAF (1.82 g, 6.96 mmol). The solution was stirred at ambient temperature for 20 minutes and then diluted with water (80 ml). The mixture was then extracted with DCM (4×40 ml), the combined organic phase was dried over magnesium sulfate and concentrated in vacuo to afford a pale yellow oil which was subjected to flash chromatography, eluent ethyl acetate/methanol 1% to afford a white solid. Single peak in LC-MS, (1.73 g, 99% purity); m/z (LC-MS, ESP), RT=2.83 mins (M+H) 411.

### (e) Methanesulfonic acid 2-{3-[2-fluoro-5-(4-oxo-3,4-dihydro-phthalazin-1-ylmethyl)-phenyl]-5-methyl-2,4-dioxo-imidazolidin-1-yl}-ethyl ester (8)

To a solution of 3-[2-fluoro-5-(4-oxo-3,4-dihydro-phthalazin-1-ylmethyl)-phenyl]-1-(2-hydroxy-ethyl)-5-methyl-imidazolidine-2,4-dione (7) (1.73 g, 4.22 mol) in anhydrous DCM (30 ml) was added triethylamine (0.95 ml, 7.0 mmol) followed by methanesulfonyl chloride (0.47 ml, 6.130 mol), the reaction mixture was stirred for 30 minutes before being diluted with water (20 ml). The mixture was extracted DCM (1×25 ml), dried over magnesium sulfate and concentrated in vacuo to afford a beige solid. Single peak in LC-MS, (1.9 g, 95% purity); m/z (LC-MS, ESP), RT=3.11 mins (M+H) 489.

### (f) Library Synthesis

To a solution of methanesulfonic acid 2-{3-[2-fluoro-5-(4-oxo-3,4-dihydro-phthalazin-1-ylmethyl)-phenyl]-5-methyl-2,4-dioxo-imidazolidin-1-yl}-ethyl ester (8) (25 mg, 0.051 mmol) in dry acetonitrile (3 ml) was added appropriate amine (0.26 mmol) and sample stirred at 40° C. for 16 hours. The reaction mixture was then subjected to preparative HPLC chromatography, to yield the compounds set out below.

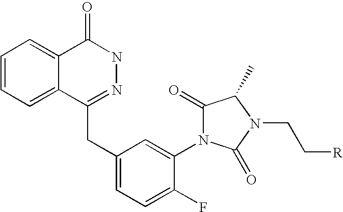

| Compound | R | M + H | RT (mins) | Purity |
|---|---|---|---|---|
| 9 | 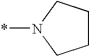 | 464.3 | 3.64* | 96 |
| 10 | 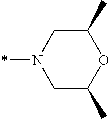 | 508.4 | 3.72* | 97 |
| 11 | 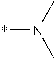 | 438.2 | 3.63* | 96 |
| 12 | 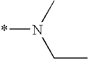 | 452.3 | 6.01 | 99 |
| 13 | 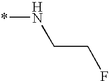 | 456.2 | 5.96 | 96 |
| 14 | 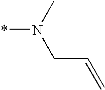 | 464.3 | 6.26 | 96 |
| 15 | 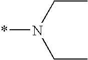 | 466.3 | 6.20 | 96 |
| 16 | 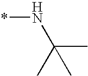 | 466.3 | 6.34 | 98 |
| 17 | 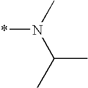 | 466.3 | 6.20 | 99 |
| 18 | 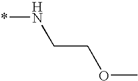 | 468.3 | 6.07 | 85 |
| 19 | 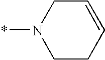 | 476.3 | 6.23 | 97 |
| 20 | 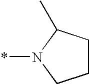 | 478.3 | 6.27 | 97 |
| 21 | 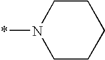 | 478.3 | 6.27 | 97 |
| 22 | 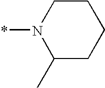 | 492.1 | 6.39 | 98 |
| 23 | 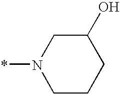 | 494.1 | 5.89 | 97 |
| 24 | 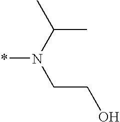 | 496.1 | 6.21 | 96 |
| 25 | 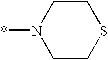 | 496 | 6.25 | 98 |
| 26 | 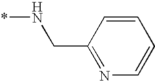 | 501.1 | 6.43 | 90 |
| 27 | 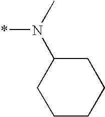 | 506.2 | 7.15 | 96 |
| 28 | 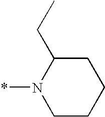 | 506.2 | 6.93 | 97 |
| 29 | 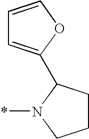 | 530.1 | 6.84 | 98 |

* = Gradient 1; all others Gradient 2

In the following examples, NMR spectra were obtained on a Bruker Avance 400 MHz NMR spectrometer equipped with a 5 mm QNP probe.

### Example 2

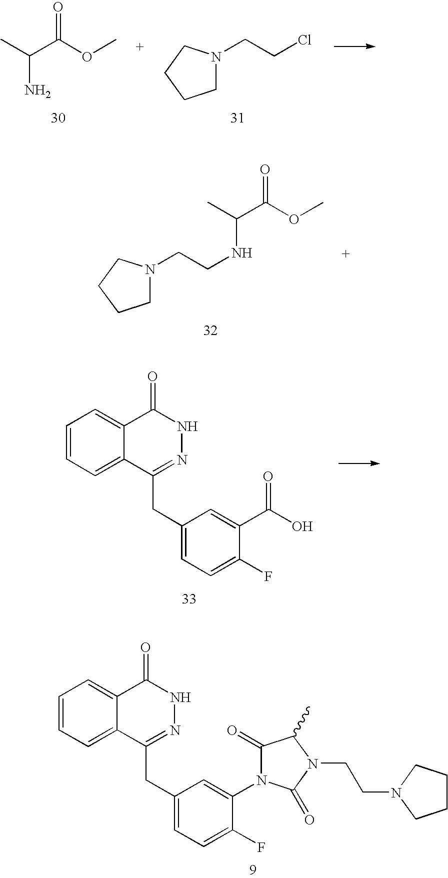

### (a) Methyl 2-(2-(pyrrolidin-1-yl)ethylamino)propanoate (32)

N,N-Diisopropylethylamine (749 ml, 4298.59 mmol) was added dropwise to DL-Alanine methyl ester hydrochloride (30)(200 g, 1432.86 mmol), 1-(2-Chloroethyl)pyrrolidine hydrochloride (31) (249 g, 1432.86 mmol) and Potassium iodide (7.60 ml, 143.29 mmol) in DMF (10 vol) (2001 ml) warmed to 80° C. over a period of 1 hour under nitrogen. The resulting slurry was stirred at room temperature for 1 day.

The reaction mixture was filtered and the solvent evaporated. The crude product was purified by flash silica chromatography, elution gradient 3 to 5% methanolic ammonia in DCM. Pure fractions were evaporated to dryness to afford methyl 2-(2-(pyrrolidin-1-yl)ethylamino)propanoate (32)(63.0 g, 21.95%) as a yellow oil. ¹H NMR (400.132 MHz, DMSO) δ 1.16 (3H, d), 1.62-1.73 (4H, m), 1.99 (1H, s), 2.30-2.61 (8H, m), 3.29 (1H, q), 3.63 (3H, s)

Compound 33 was synthesized as described in WO 2004/080976 (Compound B), which is incorporated herein by reference.

### (b) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (9)

A solution of methyl 2-(2-(pyrrolidin-1-yl)ethylamino)propanoate (32)(135 g, 613.54 mmol) in acetonitrile (1226 ml, 6.7 vol) was added dropwise to a stirred slurry of 2-fluoro-5-((4-oxo-3,4-dihydrophthalazin-1-yl)methyl)benzoic acid (33)(183 g, 613.54 mmol), and triethylamine (188 ml, 1349.79 mmol) in acetonitrile (20 vol) (3652 ml) at 85° C., over a period of 10 minutes under nitrogen. To the resulting suspension was added a solution of diphenyl phosphorazidate (145 ml, 674.90 mmol) in acetonitrile (604 ml, 3.3 vol) dropwise over 5 minutes and the reaction was allowed to stir for 1 hour. The reaction mixture was evaporated to dryness and redissolved in DCM (1830 ml, 10 vol), and washed sequentially with water (1830 ml×2, 10 vol×2), saturated NaHCO₃ (1830 ml, 10 vol), and saturated brine (1830 ml, 10 vol). The organic layer was dried over MgSO₄, filtered and evaporated to afford crude product. The crude product was purified by flash silica chromatography, elution gradient 3 to 5% methanolic ammonia in DCM. Pure fractions were evaporated to dryness to afford 3-(2-fluoro-5-((4-oxo-3,4-dihydrophthalazin-1-yl )methyl)phenyl)-5-methyl-1-(2-(pyrrolidin-1-yl)ethyl)imidazolidine-2,4-dione (9)(271 g, 95%) as a white foam. ¹H NMR (400.132 MHz, DMSO) δ 1.41 (3H, d), 1.60-1.72 (4H, m), 2.46 (4H, d), 2.55-2.66 (2H, m), 3.20-3.31 (1H, m), 3.65 (1H, t), 4.31-4.44 (3H, m), 7.34 (2H, dd), 7.46-7.53 (1H, m), 7.84 (1H, td), 7.90 (1H, td), 7.98 (1H, d), 8.27 (1H, dd), 12.62 (1H, s)

### Example 3

### (a) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione hydrochloride (9a)

(i) Hydrochloric acid (HCl in IPA 5N to 6N) (216 μl, 1.08 mmol) was added dropwise to 3-(2-fluoro-5-((4-oxo-3,4-d ihydrophthalazin-1-yl )methyl)phenyl)-5-methyl-1-(2-(pyrrolidin-1-yl)ethyl)imidazolidine-2,4-dione (9)(500 mg, 1.08 mmol), in MeOH (10 vol) (4994 μl) at 20° C. over a period of 10 minutes under nitrogen. The resulting solution was stirred overnight. The precipitate was collected by filtration, washed with MeOH (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione hydrochloride (504 mg, 93%) as a white solid, which was used without further purification. ¹H NMR (400.13 MHz, DMSO-d₆) δ 1.43 (3H, d), 1.88-2.00 (4H, m), 3.04 (2H, d), 3.26 (1H, s), 3.51-3.60 (3H, m), 4.00 (1H, s), 4.14 (1H, s), 4.34 (2H, s), 4.50 (1H, s), 7.31-7.36 (1H, m), 7.46-7.50 (2H, m), 7.82-7.86 (1H, m), 7.88-7.92 (1H, m), 7.99 (1H, d), 8.25-8.28 (1H, m), 10.81 (1H, s), 12.62 (1H, s)

(ii) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione hydrochloride obtained in step (i) (281 g, 562.04 mmol) in ethyl acetate (2810 ml, 10 vol) under nitrogen. The resulting slurry was stirred at ambient tempertaure for 5 days. The precipitate was collected by filtration, washed with Et₂O (562 ml, 2 vol) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione hydrochloride (266 g, 95%) as a white crystalline solid. ¹H NMR (400.13 MHz, DMSO-d₆) δ 1.43 (3H, d), 1.88-2.00 (4H, m), 3.04 (2H, d), 3.26 (1H, s), 3.51-3.60 (3H, m), 4.00 (1H, s), 4.14 (1H, s), 4.34 (2H, s), 4.50 (1H, s), 7.31-7.36 (1H, m), 7.46-7.50 (2H, m), 7.82-7.86 (1H, m), 7.88-7.92 (1H, m), 7.99 (1H, d), 8.25-8.28 (1H, m), 10.81 (1H, s), 12.62 (1H, s)

### (b) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione succinate (9b)

A solution of succinic acid (127 mg, 1.08 mmol) in MeOH (10 vol) (4994 μl) was added dropwise to a stirred solution of 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (9)(500 mg, 1.08 mmol), in MeOH (10 vol) (4994 μl) at 20° C., over a period of 5 minutes under nitrogen. The resulting solution was stirred overnight. The precipitate was collected by filtration, washed with TBME (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione succinate (453 mg, 72.2%) as a white solid, which was used without further purification. ¹H NMR (400.132 MHz, DMSO) δ 1.41 (3H, d), 1.66-1.71 (4H, m), 2.38 (2H, s), 2.56 (4H, s), 2.61-2.78 (2H, m), 3.28 (1H, dd), 3.68 (1H, dt), 4.33-4.45 (1H, m), 4.35 (2H, s), 7.35 (2H, dd), 7.46-7.52 (1H, m), 7.84 (1H, td), 7.90 (1H, td), 7.98 (1H, d), 8.27 (1H, dd), 12.62 (1H, s)

### (c) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione fumarate (9c)

A solution of fumaric acid (125 mg, 1.08 mmol) in MeOH (10 vol) (4994 μl) was added dropwise to a stirred solution of 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (9)(500 mg, 1.08 mmol), in MeOH (10 vol) (4994 μl) at 20° C., over a period of 10 minutes under nitrogen. The resulting solution was stirred overnight. The precipitate was collected by filtration, washed with TBME (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione fumarate (485 mg, 78%) as a white solid, which was used without further purification. ¹H NMR (400.132 MHz, DMSO) δ 1.34 (3H, d), 1.60-1.74 (4H, m), 2.51-2.94 (6H, m), 3.27 (1H, dt), 3.69 (1H, quintet), 4.27 (2H, s), 4.34 (1H, d), 6.47 (1.5H, s), 7.22-7.32 (2H, m), 7.40 (1H, ddd), 7.76 (1H, td), 7.82 (1H, td), 7.90 (1H, d), 8.19 (1H, dd), 12.55 (1H, s)

### (d) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione mesylate (9d)

Methanesulfonic acid (70.7 μl, 1.08 mmol) was added dropwise to 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (9)(500 mg, 1.08 mmol), in MeOH (10 vol) (4994 μl) at 20° C. over a period of 5 minutes under nitrogen. The resulting solution was stirred overnight. The precipitate was collected by filtration, washed with Et₂O (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione mesylate (479 mg, 79%) as a white solid. ¹H NMR (400.132 MHz, DMSO) δ 1.43 (3H, d), 1.80-1.95 (2H, m), 1.95-2.10 (2H, m), 2.33 (3H, s), 3.03-3.16 (2H, m), 3.28 (2H, d), 3.45-3.69 (3H, m), 3.89-4.03 (1H, m), 4.36 (2H, s), 4.43 (1H, d), 7.29-7.41 (2H, m), 7.50-7.57 (1H, m), 7.84 (1H, td), 7.90 (1H, td), 7.98 (1H, d), 8.27 (1H, dd), 9.43 (1H, s), 12.63 (1H, s)

### (e) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione tosylate (9e)

A solution of p-toluenesulfonic acid monohydrate (192 μl, 1.19 mmol) in MeOH (10 vol) (4994 μl) was added dropwise to a stirred solution of 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (500 mg, 1.08 mmol), in MeOH (10 vol) (4994 μl) at 20° C., over a period of 5 minutes under nitrogen. The resulting solution was stirred at overnight. The precipitate was collected by filtration, washed with Et2O (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione tosylate (527 mg, 77%) as a white solid. ¹H NMR (400.132 MHz, DMSO) δ 1.43 (3H, d), 1.79-1.93 (2H, m), 1.96-2.09 (2H, m), 2.29 (3H, s), 3.03-3.16 (2H, m), 3.22-3.32 (1H, m), 3.45-3.67 (4H, m), 3.90-4.01 (1H, m), 4.36 (2H, s), 4.41 (1H, s), 7.12 (2H, d), 7.29 (1H, s), 7.38 (1H, t), 7.48 (2H, dt), 7.51-7.59 (1H, m), 7.84 (1H, td), 7.90 (1H, td), 7.97 (1H, d), 8.27 (1H, dd), 9.32 (1H, s), 12.63 (1H, s)

### (f) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione maleate (9f)

A solution of maleic acid (125 mg, 1.08 mmol) in MeOH (10 vol) (4994 μl) was added dropwise to a stirred solution of 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (9)(500 mg, 1.08 mmol), in MeOH (10 vol) (4994 μl) at 20° C., over a period of 5 minutes under nitrogen. The resulting solution was stirred overnight. The precipitate was collected by filtration, washed with Et₂O (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione maleate (490 mg, 78%) as a white solid. ¹H NMR (400.132 MHz, DMSO) δ 1.43 (3H, d), 1.79-2.13 (4H, m), 3.19-3.41 (6H, m), 3.45-3.58 (1H, m), 3.89-4.01 (1H, m), 4.37 (2H, s), 4.38-4.47 (1H, m), 6.04 (2H, s), 7.20-7.33 (1H, m), 7.38 (1H, t), 7.52-7.60 (1H, m), 7.84 (1H, td), 7.90 (1H, td), 7.97 (1H, d), 8.27 (1H, dd), 12.63 (1H, s)

### (g) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione sulphate (9g)

Sulfuric acid (58.6 μl, 1.08 mmol) was added dropwise to 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (500 mg, 1.08 mmol) in MeOH (10 vol) (4994 μl) at 20° C. over a period of 5 minutes under nitrogen. The resulting solution was stirred overnight. The precipitate was collected by filtration, washed with Et₂O (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione sulphate (522 mg, 86%) as a white solid. ¹H NMR (400.132 MHz, DMSO) δ 1.36 (3H, d), 1.71-1.87 (2H, m), 1.88-2.03 (2H, m), 2.96-3.09 (2H, m), 3.16-3.26 (2H, m), 3.38-3.60 (3H, m), 3.82-3.94 (1H, m), 4.29 (2H, s), 4.31-4.41 (1H, m), 7.19-7.27 (1H, m), 7.30 (1H, t), 7.44-7.51 (1H, m), 7.77 (1H, td), 7.83 (1H, td), 7.91 (1H, d), 8.19 (1H, dd), 9.28 (1H, s), 12.55 (1H, s)

### (h) 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione phosphate (9h)

Phosphoric acid (73.8 μl, 1.08 mmol) was added dropwise to 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (9)(500 mg, 1.08 mmol) in MeOH (10 vol) (4994 μl) at 20° C. over a period of 5 minutes under nitrogen. The resulting solution was stirred overnight. The precipitate was collected by filtration, washed with Et₂O (5 mL) and dried under vacuum to afford 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione (422 mg, 69.7%) as a white solid. ¹H NMR (400.132 MHz, DMSO) δ 1.42 (3H, d), 1.77 (4H, s), 2.70-3.01 (6H, m), 3.31-3.42 (1H, m), 3.76 (1H, dt), 4.35 (2H, s), 4.41 (1H, d), 7.31-7.41 (2H, m), 7.49 (1H, ddd), 7.84 (1H, td), 7.90 (1H, td), 7.99 (1H, d), 8.27 (1H, dd), 12.63 (1H, s)

### Example 4

Compound 9:

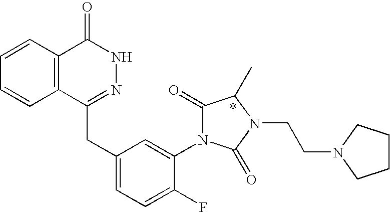

has a chiral centre where indicated. This racemic mixture was separated using chiral preparative HPLC.

This separation was carried out on a Rainin prep machine (200 ml heads) using a Merck 100 mm 20 μm Chiralpak AD column. The Eluent was a mixture of i-hexane, ethanol and methanol (70:15:15), which was flowed at a rate of 190 ml/min. The analaysis was carried out with a wavelength of 215 nm. Complete separation of the two isomers was achieved using a sample concentration of 12.5 mg/ml, an injection volumn of 40 ml and a run time of 3 hours.

However, on standing in solution compound 9 racemises.

### Example 5

In order to assess the inhibitory action of the compounds, the following assay was used to determine IC₅₀ values (Dillon, et al., JBS., 8(3), 347-352 (2003)).

Mammalian PARP, isolated from Hela cell nuclear extract, was incubated with Z-buffer (25 mM Hepes (Sigma); 12.5 mM MgCl₂ (Sigma); 50 mM KCl (Sigma); 1 mM DTT (Sigma); 10% Glycerol (Sigma) 0.001% NP-40 (Sigma); pH 7.4) in 96 well FlashPlates (TRADE MARK) (NEN, UK) and varying concentrations of said inhibitors added. All compounds were diluted in DMSO and gave final assay concentrations of between 10 and 0.01 μM, with the DMSO being at a final concentration of 1% per well. The total assay volume per well was 40 μl.

After 10 minutes incubation at 30° C. the reactions were initiated by the addition of a 10 μl reaction mixture, containing NAD (5 μM), ³H-NAD and 30mer double stranded DNA-oligos. Designated positive and negative reaction wells were done in combination with compound wells (unknowns) in order to calculate % enzyme activities. The plates were then shaken for 2 minutes and incubated at 30° C. for 45 minutes.

Following the incubation, the reactions were quenched by the addition of 50 μl 30% acetic acid to each well. The plates were then shaken for 1 hour at room temperature.

The plates were transferred to a TopCount NXT (TRADE MARK) (Packard, UK) for scintillation counting. Values recorded are counts per minute (cpm) following a 30 second counting of each well.

The % enzyme activity for each compound is then calculated using the following equation:

$$\% Inhibition = 100 - ( 100 \times \frac{( c p m of unknowns - mean negative c p m )}{( mean positive c p m - mean negative c p m )} )$$

IC₅₀ values (the concentration at which 50% of the enzyme activity is inhibited) were calculated, which are determined over a range of different concentrations, normally from 10 μM down to 0.001 μM. Such IC₅₀ values are used as comparative values to identify increased compound potencies.

Compounds 9 to 11 had a mean IC₅₀ of less than 0.1 μM.

The mean IC₅₀ values for the compounds are presented below:

| Compound | Mean IC₅₀ (μM) |
|---|---|
| 9 | 0.0038 |
| 10 | 0.0031 |
| 11 | 0.0033 |
| 12 | 0.0030 |
| 13 | 0.0031 |
| 14 | 0.0035 |
| 15 | 0.0030 |
| 16 | 0.0029 |
| 17 | 0.0040 |
| 18 | 0.0038 |
| 19 | 0.0040 |
| 20 | 0.0032 |
| 21 | 0.0026 |
| 22 | 0.0029 |
| 23 | 0.0046 |
| 24 | 0.0036 |
| 25 | 0.0028 |
| 26 | 0.0037 |
| 27 | 0.0030 |
| 28 | 0.0042 |
| 29 | 0.0039 |

The Potentiation Factor (PF₅₀) for compounds is calculated as a ratio of the IC₅₀ of control cell growth divided by the IC₅₀ of cell growth+PARP inhibitor. Growth inhibition curves for both control and compound treated cells are in the presence of the alkylating agent methyl methanesulfonate (MMS). The test compounds were used at a fixed concentration of 0.2 or 0.5 micromolar. The concentrations of MMS were over a range from 0 to 10 μg/ml. Cell growth was assessed using the sulforhodamine B (SRB) assay (Skehan, P., et al., (1990) New calorimetric cytotoxicity assay for anticancer-drug screening. J. Natl. Cancer Inst. 82, 1107-1112). 2,000 HeLa cells were seeded into each well of a flat-bottomed 96-well microtiter plate in a volume of 100 μl and incubated for 6 hours at 37° C. Cells were either replaced with media alone or with media containing PARP inhibitor at a final concentration of 0.5, 1 or 5 μM. Cells were allowed to grow for a further 1 hour before the addition of MMS at a range of concentrations (typically 0, 1, 2, 3, 5, 7 and 10 μg/ml) to either untreated cells or PARP inhibitor treated cells. Cells treated with PARP inhibitor alone were used to assess the growth inhibition by the PARP inhibitor.

Cells were left for a further 16 hours before replacing the media and allowing the cells to grow for a further 72 hours at 37° C. The media was then removed and the cells fixed with 100 μl of ice cold 10% (w/v) trichloroacetic acid. The plates were incubated at 4° C. for 20 minutes and then washed four times with water. Each well of cells was then stained with 100 μl of 0.4% (w/v) SRB in 1% acetic acid for 20 minutes before washing four times with 1% acetic acid. Plates were then dried for 2 hours at room temperature. The dye from the stained cells was solubilized by the addition of 100 μl of 10 mM Tris Base into each well. Plates were gently shaken and left at room temperature for 30 minutes before measuring the optical density at 564 nM on a Microquant microtiter plate reader.

At 1 nM, compounds 9, 12, 19, 20, 21 and 22 had a mean PF₅₀ of greater than 2. At 30 nM, compounds 9, 10, 12, 20, 21, 22 and 23 had a mean PF₅₀ of greater than 2. At 200 nM, compounds 13, 14, 16, 17, 18, 24, 26, 27, 28 and 29 had a mean PF₅₀ of greater than 2.

Solubility Assay

A typical assay that may be used to assess the solubility of the compounds of the present invention is as follows. The solubility of the compound is assessed in water and phosphate-buffered saline (pbs) at pH 7.4. The samples are all allowed to equilibriate in the solvent (with shaking) for 20 hours at room temperature. After that period, the samples will be visually examined to determine the presence/absence of un-dissolved solid. The samples will be centrifuged or filtered as necessary to remove insoluble material, and the solution analysed to determine solubility of the DS, diluting both aqueous and DMSO samples to a similar concentration with DMSO. The area of the peak obtained by HPLC (using the diode array detector) from the sample will be compared to the area of the peak from the DMSO solution (diluted to the same concentration as the sample) and quantified taking into account the weight of sample taken for initial dissolution. The assumption is made that the sample will be completely soluble in DMSO at the levels used for testing.

Comparing the ratio of the peak areas, and knowing the concentration of the original samples, the solubility may be calculated.

Preparation of Samples

About 1 mg of the sample is weighed accurately into a 4-ml glass vial and exactly 1.0 ml of water, aqueous buffer or DMSO, is added to it by pipette. Each vial is ultrasonicated for up to 2 minutes to assist solublisation of the solid. The samples are retained at room temperature for 20 hours, shaking on an orbital shaker. The vials are examined after this period to determine the presence/absence of un-dissolved solid. The samples should be centrifuged, or filltered through a 0.45 μm filter, to remove insoluble material if necessary, and the filtrate analysed to determine concentration of the compound in solution, after diluting all samples as appropriate with DMSO. 20 μl is injected onto the HPLC using the method shown below, injecting all samples in duplicate. The maximum solubility that can be determined using this method is nominally 1.0 mg/ml, the weight taken divided by the volume of solvent used.

Analytical Techniques

The samples are subjected to LC/MS using a Waters Micromass ZQ instrument (or equivalent) with test parameters typically as follows.

Waters Micromass ZQ in positive ion mode.

Scanning from m/z 100 to 800

Mobile phase A—0.1% aqueous formic acid

Mobile phase B—0.1% formic acid in Acetonitrile

Column—Jones Chromatography Genesis 4μ C18 column, 4.6×50 mm

Flow rate 2.0 ml/min

Injection volume 30 μl injection into a 20 μl loop.

Gradient—starting at 95% A/5% B, rising to 95% B after 4 minutes, holding there for four minutes, then back to the starting conditions. (This may be modified if necessary to obtain better separation of peaks).

PDA detection scanning from 210 to 400 nm

Quantification of Samples

Initial examination of the sample vials containing the aqueous dilution indicates whether or not the compound is soluble in that buffer at that concentration. If it is not soluble, this should be reflected in the concentration obtained in solution by HPLC/MS. If the solution is clear, then the concentration in aqueous solvent should be similar to that in DMSO, unless degradation of the compound has occurred; this should be visible on the chromatogram.

The assumption is made that the samples will be completely soluble in DMSO, therefore the peak size obtained from that sample will reflect 100% solubility. Assuming that the dilutions of all samples have been the same, then solubility in mg/ml=(area from pbs solution/area from DMSO solution)×(original weight in DMSO solution/dilution).

Stability Assay

A typical assay that may be used to assess the stability of the compounds of the present invention is as follows. The stability of the compounds is assessed in various aqueous solutions and phosphate-buffered saline (pbs). The samples will be tested at nominal pH 2, 7.4 (pbs) and 9. These values are chosen to reflect the conditions encountered in the gut during digestion (about pH 2 up to about pH 9), and in blood plasma (nominal pH 7.4). The samples are dissolved in methanol/DMSO to prepare a stock solution. The stock solution is then diluted to give aqueous solutions at a nominal pH of 2, 7.4 and 9. Samples are analysed immediately to give initial values for purity and possible related compounds. The samples are then retained at (usually) room temperature, and re-analysed after 2 hours, 6 hours, 24 hours and 2 days (nominal).

The stability of the compounds in this aqueous buffer over the period of the test can be assessed by comparison of the chromatogram of the sample at initial with that in aqueous buffer after the given time period.

Preparation and Analysis of Samples

About 5-6 mg of the sample is accurately measured into a 4-ml glass vial and approximately 2 mls of methanol is added to it. If solution is not complete in this organic solvent, a further 0.5-1.0 ml of DMSO is added; the final solution strength should be about 2.0 mg/ml. This 2 mg/ml organic solution is then diluted 1+3 with (a) water, to use as the ‘initial’ sample, (b) very dilute HCl at about pH 2, (c) pbs at pH 7.4, and (d) very dilute NaOH at about pH 9. The pH of each dilution is then checked and noted; if not close to the desired value, the pH may be adjusted with dilute acid or alkali, as appropriate. These dilutions are made at intervals after the ‘initial’ sample, to allow for the delay due to the HPLC analysis. All samples should be diluted 50/50 with DMSO prior to injection onto the HPLC.

The samples are retained at room temperature for 2 hours initially, then sub-samples as above diluting 50/50 with DMSO prior to injection. 20 μl is injected onto the HPLC using the method shown below, injecting all samples in duplicate. The above is repeated after 6 hours, 24 hours and 2 days (nominal time intervals)

Analytical Techniques

The samples will be subjected to LC/MS using a Waters Micromass ZQ instrument (or equivalent) with test parameters typically as follows.

Waters Micromass ZQ in positive ion mode.

Scanning from m/z 150 to 900

Mobile phase A—0.1% aqueous formic acid

Mobile phase B—0.1% formic acid in Acetonitrile

Column—Jones Chromatography Genesis 4μ C18 column, 4.6×50 mm

Flow rate 2.0 ml/min

Injection volume 30 μl injection into a 20 μl loop.

Gradient—starting at 95% A/5% B, rising to 95% B after 5 minutes, holding there for four minutes, then back to the starting conditions. (This may be modified if necessary to obtain better separation of peaks).

PDA detection scanning from 210 to 400 nm

Assessment of Stability

The chromatogram peak areas of the samples at the various pH's are compared after any given time interval with those from the initial analysis at time zero. The DS peak should be quantified as a percentage of the initial sample, and the values tabulated.

VC8 assay

In order to assess the growth inhibitory action of compounds on BRCA2 deficient (VC8−hamster line) and BRAC2 complemented (VC8+BAC) cells the following assay was used to determine GI₅₀ values.

500 VC8 cells or 200 VC8+BAC cells were seeded into each well of a flat-bottomed 96-well microtiter plate in a volume of 90 μl and incubated for 4-6 hours at 37° C. All compounds were diluted in media (Dulbecco's Modified Eagle's Medium (DMEM),10% Fetal Bovine Serum, Penicillin/Sretptomycin/Glutamine) and added to the cells at final concentrations of between 0 and 300 nM.

Cells were left for a further 48 hours before replacing the media with fresh media (no compound) and allowing the cells to grow for a total of 120 hours at 37° C. The medium was then removed and the cells fixed with 50 μl of ice cold 10% (w/v) tricholoracetic acid. The plates were incubated at 4° C. for 30 minutes and then washed three times with water. Each well of cells was then stained with 50 μl of 0.4% (w/v) sulforhodamine B (SRB) in 1% acetic acid for 15 minutes before washing three times with 1% acetic acid. Plates were then dried for 2 hours at room temperature. The dye from the stained cells was solubilised by the addition of 100 μl of 10 mM Tris Base into each well. Plates were then shaken and the optical density at 564 nM was measured on a Microquant microtiter plate reader.

The GI₅₀ is calculated as the pM concentration of compound required to inhibit 50% of cell growth.

### Example 6

3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione hydrochloride (9a) as obtained above was studied further by measuring its solid state properties as set out below.

X-Ray Powder Diffraction

TABLE A

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with fixed slits

Analytical Instrument: Siemens D5000.

The X-ray powder diffraction spectra were determined by mounting a sample of the crystalline material on a Siemens single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 degrees 2-theta in theta-theta mode. The running time was 31 minutes and 41 seconds. The instrument was equipped with a scintillation counter as detector. Control and data capture was by means of a Dell Optiplex 686 NT 4.0 Workstation operating with Diffract+ software. Persons skilled in the art of X-ray powder diffraction will realise that the relative intensity of peaks can be affected by, for example, grains above 30 microns in size and non-unitary aspect ratios that may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values.

Differential Scanning Calorimetry

Analytical Instrument: TA Instruments Q1000 DSC.

Typically less than 5 mg of material contained in a standard aluminium pan fitted with a lid was heated over the temperature range 25° C. to 325° C. at a constant heating rate of 10° C. per minute. A purge gas of nitrogen was used—flow rate 100 ml per minute.

Thermal Gravimetric Analysis

Analytical instrument: TA Instruments Q5000TGA

Typically less than 10 mg of material contained in a standard platinum pan was heated from ambient to 325° C. at a constant heating rate of 10° C. per minute. A purge gas of nitrogen was used—flow rate 25 ml per minute.

The X-Ray Powder Diffraction Pattern is shown in FIG. 1. The ten most prominent peaks are listed in table B below:

TABLE B

| Angle 2- | | Relative |
|---|---|---|
| Theta (2θ) | Intensity % | Intensity |
| 11.6 | 100 | vs |
| 24.6 | 93.0 | vs |
| 26.4 | 92.5 | vs |
| 14.9 | 88.4 | vs |
| 26.8 | 81.7 | vs |
| 20.6 | 69.9 | vs |
| 17.4 | 65.6 | vs |
| 23.1 | 58.9 | vs |
| 9.7 | 48.7 | vs |
| 25.0 | 44.6 | vs |

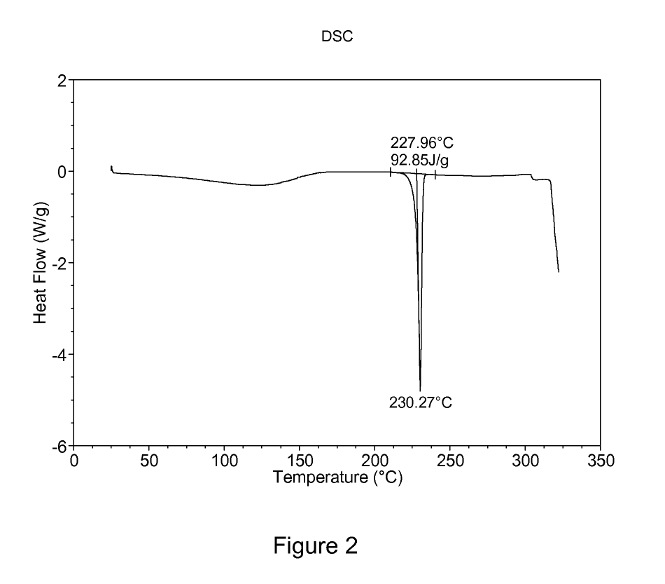

The DSC thermogram is shown in FIG. 2. This shows an initial broad event from ambient to 150° C., followed by a subsequent melt with an onset of 228° C. and peak at 230° C.

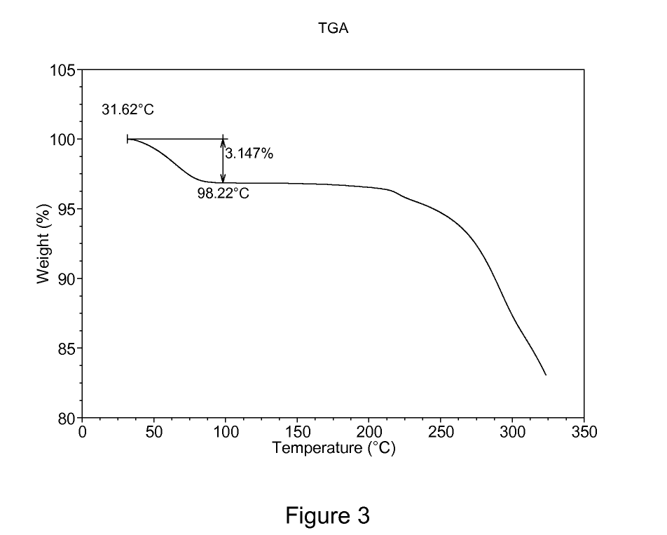

The TGA thermogram is shown in FIG. 3. This shows a weight loss from ambient to 100° C. of 3.13% w/w, consistent with the loss of a molecule of water.

Without wishing to be bound by theory, it is thought the DSC and TGA analyses show the loss of water from the material 9a followed by the melting of the dehydrated form.

1. A compound of the formula (I): 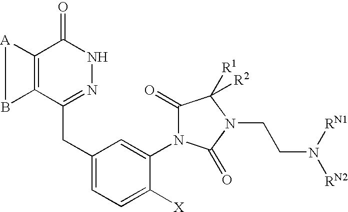
wherein:
A and B together represent an optionally substituted, fused aromatic ring;
X is selected from H and F;
R¹ and R² are independently selected from H and methyl;
R^{N1} is selected from H and optionally substituted C₁₋₇ alkyl;
R^{N2} is selected from H, optionally substituted C₁₋₇ alkyl, C₃₋₇ heterocyclyl and C₅₋₆ aryl;
or R^{N1} and R^{N2} and the nitrogen atom to which they are bound form an optionally substituted nitrogen containing C₅₋₇ heterocyclic group.

2. A compound according to claim 1, wherein A and B together represent a fused benzene or pyridine ring.

3. A compound according to claim 1, wherein X is F.

4. A compound according to claim 1, wherein R¹ is H and R² is methyl.

5. A compound according to claim 1, wherein R^{N1} is optionally substituted C₁₋₇ alkyl.

6. A compound according to claim 5, wherein R^{N1} is selected from methyl, ethyl, cyclopropyl, iso-propyl, tert-butyl, 2,2-dimethylpropyl, cyclobutyl, cyclohexyl, which is optionally substituted by a group selected from halo, hydroxy, alkoxy and C₅₋₆ aryl.

7. A compound according to claim 1, wherein R^{N2} is C₁₋₇ alkyl.

8. A compound according to claim 7, wherein R^{N2} is selected from for example, methyl, ethyl, cyclopropyl, iso-propyl, tert-butyl, 2,2-dimethylpropyl, cyclobutyl, cyclohexyl, which is optionally substituted by a group selected from halo, hydroxy, alkoxy and C₅₋₆ aryl.

9. A compound according to claim 1, wherein R^{N2} is C₃₋₇ heterocylcyl, optionally substituted with a group selected from C₁₋₇ alkyl, halo, hydroxy, alkoxy and amino.

10. A compound according to claim 1, wherein R^{N2} is C₅₋₆ aryl, optionally substituted with a group selected C₁₋₇ alkyl, halo, hydroxy, alkoxy and amino.

11. A compound according to claim 1, wherein R^{N1} and R^{N2} are the same.

12. A compound according to claim 11, wherein R^{N1} and R^{N2} are selected from unsubstituted C₁₋₇ alkyl.

13. A compound according to claim 1, wherein R^{N1} and R^{N2} and the nitrogen atom to which they are bound form an optionally substituted nitrogen containing C₅₋₇ heterocyclic group.

14. A compound according to claim 13, wherein R^{N1} and R^{N2} and the nitrogen atom to which they are bound form a group selected from pyrolidine, piperidine, morpholine and thiomorpholine.

15. A compound according to claim 13, wherein the C₅₋₇ heterocyclic is substituted by substituents selected from C₁₋₇ alkyl, C₅₋₆ aryl, hydroxy and C₁₋₇ alkyoxy.

16. A compound according to claim 13, wherein the C₅₋₇ heterocyclic is unsubstituted.

17. 3-(2-fluoro-5-((4-oxo-3,4-dihydrophthalazin-1-yl)methyl)phenyl)-5-methyl-1-(2-(pyrrolidin-1-yl)ethyl)imidazolidine-2,4-dione, and isomers, pharmaceuticaly acceptable salts and solvates thereof.

18. A pharmaceutical composition comprising a compound according to claim 1 and a pharmaceutically acceptable carrier or diluent.

19. A method of treatment of disease ameliorated by the inhibition of PARP, comprising administering to a subject in need of treatment a therapeutically-effective amount of a compound according to claim 1.

20. A method according to claim 19, wherein the method of treatment is
(a) preventing poly(ADP-ribose) chain formation by inhibiting the activity of cellular PARP (PARP-1 and/or PARP-2);
(b) the treatment of: vascular disease; septic shock; ischaemic injury, both cerebral and cardiovascular; reperfusion injury, both cerebral and cardiovascular; neurotoxicity, including acute and chronic treatments for stroke and Parkinsons disease; angiogenesis; haemorraghic shock; inflammatory diseases, such as arthritis, inflammatory bowel disease, ulcerative colitis and Crohn's disease; multiple sclerosis; secondary effects of diabetes; as well as the acute treatment of cytoxicity following cardiovascular surgery or diseases ameliorated by the inhibition of the activity of PARP;
(c) use as an adjunct in cancer therapy or for potentiating tumour cells for treatment with ionizing radiation or chemotherapeutic agents.

21. A method of treatment of cancer, comprising administering to a subject in need of treatment a therapeutically-effective amount of a compound according to claim 1 in combination simultaneously or sequentially with radiotherapy (ionizing radiation) or chemotherapeutic agents.
